(19) United States
(12) Patent Application Publication
(10) Pub. No.: US 2007/0166254 A1
(43) Pub. Date: Jul. 19, 2007

(54) ANTIPERSPIRANT STICK COMPOSITIONS

(75) Inventor: James Michael Bianchi, Chicago, IL (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: CONOPCO, INC., d/b/a UNILEVER, Englewood Cliffs, NJ (US)

(21) Appl. No.: 11/622,682

(22) Filed: Jan. 12, 2007

Related U.S. Application Data
(60) Provisional application No. 60/758,895, filed on Jan. 14, 2006.

(51) Int. Cl. A61K 8/28; A61K 8/89

(52) U.S. Cl. 424/66

(57) ABSTRACT

Disclosed are antiperspirant stick compositions comprising specified amounts of antiperspirant active; carrier oil comprising volatile silicone oil and non-volatile masking oil; structurant comprising fatty alcohol and cosmetically acceptable wax having a melting point of 75 to 95° C., a portion of wax comprises polyethylene in specified amounts. Also disclosed are methods of making such stick compositions, and methods of ameliorating perspiration by the topical application of such compositions to the skin.

### BACKGROUND OF THE INVENTION

Solid sticks are a widely used form of antiperspirant products. Such products commonly comprise antiperspirant active, volatile carrier oil, and structurant. Of particular interest as structurants are fatty alcohols, in particular, C₁₆-C₂₄ linear fatty alcohols. In addition to providing desirable structuring properties, fatty alcohols are generally easy to process. Fatty alcohols can also provide good sensory properties, e.g., glide, and easy wash-off, to the resulting sticks. A fatty alcohol widely used in many antiperspirant sticks is stearyl alcohol.

While fatty alcohols have properties that make their use advantageous, fatty alcohol-containing antiperspirant sticks tend to leave a white residue on the skin after use. When dry, antiperspirant actives generally exist as white powders; thus, they too can contribute to the deposition of a visible residue on skin or clothes. Providing a fatty alcohol-containing antiperspirant stick which leaves little or no white residue on skin or clothes poses a significant challenge to formulators, particularly in the case of sticks containing fatty alcohols at levels of 12% and above, as whitening tends to be exacerbated as the fatty alcohol content of a stick is increased.

Numerous routes to producing low residue, fatty alcohol-containing antiperspirant sticks have been disclosed in the art, many of which focus on the incorporation of one or more non-volatile carrier oils of a type known as “masking agents”, exemplary of which are non-volatile oils from chemical classes that include polyalkylene glycol alkyl ethers, non-volatile silicones, non-volatile organic esters, and non-volatile aliphatic hydrocarbons.

U.S. Pat. No. 5,531,986 discloses antiperspirant solid stick compositions that are therein said to have “low (substantially no) visible (white) residue on the skin after application and after drying”. Disclosed as illustrative of such compositions are compositions that include, based on the total weight of the composition: (1) 10-60%, preferably 30-40%, volatile silicone material; (2) 5.01-50%, most preferably 5.01-15%, non-volatile silicone material; (3) 1-15% dimethicone copolyol; (4) 2-10% high melting point wax; (5) 2-30% low melting point wax; and (6) 10-30% antiperspirant active. At column 2, lines 39 to 48, the patent states: “By providing the antiperspirant solid stick composition (e.g., silicone-based antiperspirant solid stick composition) having volatile and non-volatile silicone materials, dimethicone copolyol and high and low-melting-point waxes, a composition leaving substantially no visible residue when applied to the skin and after drying is achieved. Moreover, such composition has good cosmetic feel on application and good feel after drying, is non-sticky and non-tacky after drying, easily glides on during application, and can be easily manufactured.” Cyclomethicone is among the volatile-silicone materials therein disclosed. Castor wax, paraffin and other natural and petroleum-based waxes are among the materials identified as high melting point waxes; fatty-acids, fatty alcohols, fatty acid esters and fatty acid amides are included among the low melting point waxes therein disclosed. The patent discloses that the compositions may desirably include inert filler materials and emollients to improve cosmetic attributes, with PPG-14 butyl ether being identified as illustrative of suitable emollients.

U.S. Pat. No. 5,833,964 discloses antiperspirant stick compositions that are said to exhibit substantially no visible residue (whitening) upon application to the skin or after drying. The compositions therein disclosed include gelled or solid compositions comprising: (a) an antiperspirant active; (b) a gelling agent, in an amount sufficient to provide the composition as a stick product (c) a vehicle for the gelling agent in an amount such that the gelling agent can dissolve therein and can gel therefrom; and (d) an emollient, including both at least one non-volatile silicone material and at least one non-volatile emollient material that is not a silicone material, wherein (i) both the at least one non-volatile silicone material and the at least one emollient material that is not a silicone material have a refractive index of at least 1.4460; and (ii) these emollient materials, as a whole, are included in an amount so as to reduce or eliminate the whitening effect of an antiperspirant active ingredient on the skin. Among the materials that are disclosed to have a refractive index of at least 1.4460 are: C₁₂-C₁₅ alkyl benzoate and PPG 14 butyl ether. At column 3, lines 25 to 32 the patent states: “By incorporating the emollient material that is not a silicone material, such emollient material having the specified refractive index, especially in combination with the non-volatile silicone material, a relatively low-cost component can be included to mask any whitening effect, while also providing improved cosmetic properties due to its emolliency effects.” Given as Example A of the patent is a formulation containing: 37% cyclomethicone, 13.1% PPG 14 butyl ether, 5.0% phenyltrimethicone, 20.0% aluminum zirconium tetrachlorohydrex glycine complex, 2.0% PEG distearate, 1.9% fragrance/starch, 4.0% hydrogenated castor oil, and 17.0% stearyl alcohol.

U.S. Pat. No. 5,449,511 to Coe discloses an anhydrous antiperspirant product that includes a non-aqueous carrier vehicle and antiperspirant active salt suspended in particle form in the carrier vehicle; and a nonvolatile, water-soluble, liquid (at 25° C.) masking agent that “interacts with the antiperspirant active to essentially eliminate discernible whitening without substantially inhibiting the antiperspirant activity of the salt when the product is applied to the skin.” See column 1, lines 44 to 52. Included among the disclosed masking agents is PPG-10 butane diol. The patent discloses that the compositions may include other compatible emollients, surfactants, preservatives, and fragrances, noting that “additionally supplemental emollients generally range from about one to about ten percent of the product.” See column 3, lines 11 to 14. Among the materials identified as supplemental emollients are PPG-14 butyl ether and C₁₂₋₁₅ alcohol benzoates. The composition set forth in Example 1 of the patent contains 44.5% cyclomethicone, 22.7% aluminum zirconium tetrachlorohydrex-glycine, 15.65 stearyl alcohol, 4.8% PPG-10 butane diol, 3.8% C12-C15 alcohol benzoate, 2.8% hydrogenated castor oil, 2.4% fragrance/starch/polyol, 1.9% myristyl myristate, 0.9% PEG-8 distearate, and 0.5% fragrance. U.S. 2004/0197281 to Walling et al. discloses an antiperspirant stick product comprising: (a) from about 5% to about 35% by weight of an antiperspirant active, (b) from about 5% to about 35% by weight of a structurant, (c) from about 20% to about 80% of a volatile fluid, and (d) 10% or less of a non-volatile organic fluid; wherein the stick has hardness of at least about 600 gram-force and an adhesion value of at least 33%, and wherein the stick is free of non-volatile silicone fluids. Compositions exemplified by the patent (Examples 1 to 6) contain 45.3-48.8% cyclopentasiloxane, 24% Al—Zr trichlorohydrex, 15-18% stearyl alcohol, 3-5.75% hydrogenated castor oil, 0.2% behenyl alcohol, 0-0.25% silica, 1-5% PPG-14 butyl ether, 0.75-2.5% mineral oil, 0-3.5% petrolatum, 0-1% myristyl myristate, and 5.25-9.5% non-volatile organic fluid. At paragraph 0063 the application lists as non-limiting examples of nonvolatile organic fluids: mineral oil, PPG-14 butyl ether, isopropyl myristate, petrolatum, butyl stearate, cetyl octanoate, butyl myristate, myristyl myristate, C₁₂-₁₅ alkyl benzoate, octyldodecanol, isostearyl isostearate, octododecyl benoatent, isostearyl lactate, isostearyl palmitate, and isobutyl stearate. The patent application discloses that the structurant can be selected from the group consisting of stearyl alcohol and other fatty alcohols, hydrogenated castor oil, paraffin wax, beeswax, carnauba, candelilla, spermaceti wax, ozokerite, ceresin, bayberry, synthetic waxes, such as Fisher-Tropsch waxes and microcrystalline wax, polyethylenes with molecular weight of about 200 to about 1000 daltons, solid triglycerides, and any mixtures thereof.

U.S. 2003/0113282 to Buranachokpaisan discloses what are therein termed “low residue anhydrous antiperspirant stick compositions.” The compositions are said to comprise: a) from about 25% to about 55%, preferably 27.5% to 35% of a volatile material, b) from about 5% to about 35%, preferably about 10 to about 30% of non-volatile liquid emollients, c) from about 0.5 to about 15%, preferably from about 1 to about 10% of non-liquid organic ester emollients having melting points between 25 and 60° C., d) from about 5% to about 20%, preferably from about 10% to about 19%, of organic wax having low or high melting points, or mixtures thereof, e) from about 0.05 to about 5% of, preferably about 0.1% to about 4% of low molecular weight polyethylene homo- or copolymer, and f) from about 15% to about 30%, preferably about 17.5 to about 27.5% of particulate antiperspirant active agent. The application exemplifies formulations that contain: 22% AlZr tetrachlorohydrex glycine, 30.4-33% cyclopentasiloxane, 12-14% stearyl alcohol, 4% hydrogenated castor oil, 10% octyl isononanoate, 2-7% isostearyl behenate, 1-1.5% polyethylene (Performalene 400), 0-4.5% talc, 2% PEG-8 distearate, 0-5% PPG-14 butyl ether, 5-6% phenyl trimethicone, 0.5% silica, 0-0.5% PEG 25 propylene glycol stearate, and 0-0.6% fragrance.

U.S. Pat. No. 6,428,777 to Boyle et al. discloses what are therein termed low residue water-containing solid antiperspirant sticks. The sticks are said to comprise: from about 5% to about 35% of a particulate antiperspirant active; from about 10% to about 60% of a volatile solvent; from about 5% to about 30% of a structurant selected from waxes and mixtures of waxes, having a melting point of at least about 40° C., from about 0.5% to about 7% of a clay as therein more particularly described; from about 0.2 to about 1.5% of an activator for the clay; and from 0.5% to about 8% water. Included among the materials discloses as suitable structurants are waxes and mixtures of waxes having a melting point of at least about 40° C., such as fatty C₁₄-C₄₀ alcohols (e g. stearyl alcohol), hydrogenated castor oil, hydrogenated vegetable oil, and polyethylene. The compositions are said to exhibit “substantially less visible residue (whitening) upon application to the skin or after drying” and further to exhibit “improved aesthetics and superior cosmetic properties.” Exemplified in the patent are compositions (A through E) that contain: 26.00 to 31.50% cyclomethicone, 24.00% aluminum zirconium tetrachlorohydrex glycine complex, 0-7.00% Bentone Gel VS5V, 0-5.00% quaternium-hectorite-18, 0-0.75% SDA-40 alcohol, 0-6.00% PPG-14 butyl ether, 0-11.0% C₁₂₋₁₅ alkyl benzoate, 0-17.00% hydrogenated polydecene, 0-2.5% isocetyl alcohol, 0-18.00% stearyl alcohol, 0-14.00% cetyl alcohol, 0-5.00% hydrogenated castor oil, 0-1.50% propylene carbonate, 0-5.00% aluminum starch octenylsuccinate, 0-2.00% talc, 0-1.50% trimethylpentanediol/adipic acid/isononanoic acid copolymer, 3.00% water soluble botanical extract (water solution), and 0.50% fragrance.

Masking agents tend to impair certain from sensory properties, resulting in sticks that can have a sticky or greasy feel. Maintaining desirable sensory properties generally becomes more difficult as the level of masking agent is increased. Additionally, sticks containing masking agent at levels above 30 wt % can be less thermally stable and may be more susceptible to dome collapse when exposed to elevated temperatures.

There remains a need for non-whitening fatty alcohol-containing antiperspirant sticks, particularly sticks containing fatty alcohol at levels of 12% by weight or above, more particularly, 15 wt % or above. Also desired are non-whitening fatty alcohol-containing sticks that have desirable antiperspirant payouts, while providing a “clean”, non-sticky feel. There also remains a need for a non-whitening fatty-alcohol containing stick that provides desirable payout as well as being thermally stable and resistant to dome collapse at temperatures of 50° C. Non-whitening sticks that are resistant to rub-off, while having desirable wash-off characteristics are also desired. Further, it is desired to provide a non-whitening fatty-alcohol-containing antiperspirant stick wherein the amount of masking oil is minimized.

Accordingly, it is an object of the present invention to provide an antiperspirant stick composition that overcomes or ameliorate one or more of the issues disclosed above.

### SUMMARY OF THE INVENTION

It has been found that in antiperspirant stick compositions comprising antiperspirant active, volatile carrier oil, and fatty alcohol, the fatty alcohol being present in the composition at a level of at least 12 wt. %, based on the total weight of the composition, the inclusion of non-volatile carrier oil at specific levels, together with cosmetically acceptable wax having a melting point of from 75 to 95° C., at least a portion of which comprises polyethylene, the polyethylene being present in the stick composition in specified amounts, provides compositions meeting one or more of the objects described above.

Accordingly, in one embodiment of this invention there is provided an antiperspirant stick composition comprising:

a) from 15 to 30 wt. % antiperspirant active;

b) carrier oil comprising:
i) volatile silicone oil, the volatile silicone oil being present in the stick composition in an amount of from 20 to 40 wt. %;
ii) non-volatile, non-silicone masking oil, the non-volatile, non-silicone masking oil being present in the stick composition in an amount of from 20 to 40wt. %;

c) structurant comprising:
i) fatty alcohol, the fatty alcohol being present in the stick composition in an amount of from 12 to 25 wt. %;
ii) cosmetically acceptable wax having a melting point of from 75 to 95° C., a portion of which comprises polyethylene, the cosmetically acceptable wax being present in the stick composition in an amount of from 2 to 10 wt. %, preferably 3 to 8% and the polyethylene being present in the stick composition in an amount of from 0.3 to 3%, preferably from 0.7 to 3%.

In another embodiment, there is provided a method of ameliorating perspiration by the topical application to the skin, of an antiperspirant active, by means of an antiperspirant stick composition according to this invention.

In yet another embodiment, there is provided a method of producing a stick composition according to this invention, which method comprises the steps of:
a) forming, at an elevated temperature, a fluid mixture comprising the antiperspirant active suspended in the carrier oil in which the structurant is dispersed and/or dissolved;
b) cooling or permitting the fluid mixture to cool to a temperature at which the mixture sets to form the stick composition.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about”. All amounts are by weight of the final composition, unless otherwise specified.

The antiperspirant stick compositions contemplated herein are in the form of solids that are characterized by their retaining their shape without lateral support under the influence of the Earth's gravity, at temperatures up to at least 50° C. The hardness of the sticks can be measured in a needle penetration test. Pursuant to this test, as the sticks become more soft, their needle penetration hardness values increase, with higher hardness values being indicative of softer sticks. In one embodiment of interest it is desirable that the antiperspirant stick compositions have hardness values of from 5 to 15 mm, more particularly 5 to 10, measured pursuant to the Hardness Method set forth below. In another embodiment of interest it is desirable that the antiperspirant stick compositions have hardness values of from 8 to 15 mm, preferably 8 to 10 mm, measured pursuant to the Hardness Method set forth below. Hardness values within these ranges are indicative of self-supporting solid sticks that are somewhat on the soft side compared to many commercially available stearyl alcohol-containing sticks. Achieving a non-whitening stick at these hardness values is of particular interest, as softer sticks generally payout at higher levels, leaving more a visible residue on the skin surface.

Desirably, the antiperspirant sticks of this invention are formulated as anhydrous compositions. As used herein “anhydrous” means that the stick composition either contains no free water, or that if free water is present, the amount thereof is not more than 1%, based on the total weight of the stick composition. In the practice of this invention it is particularly desirable that, if free water is present, the amount thereof is less than 0.5% of the total weight of the stick composition. Bound or complexed water, as, for example, water of hydration in the antiperspirant salt, is deemed not to be free.

Antiperspirant Active

Exemplary of the antiperspirant actives used herein are one or more aluminum, zirconium and mixed aluminum/zirconium salts, optionally complexed. Preferred aluminum, zirconium and aluminum/zirconium salts contain a halide, especially chloride and especially preferred salts are basic salts, which is to say a fraction of the halide within the empirical formula has been replaced by bound hydroxyl groups. Halohydroates, particularly chlorohydrate salts are very highly desired.

Aluminum halohydrates are usually defined by the general formula Al₂(OH)ₓQ_{y}.wH₂O in which Q represents chlorine, bromine or iodine, x is variable from 2 to 5 and x+y=6 while wH₂O represents a variable amount of hydration. Aluminum chlorohydrate as made comprises a mixture of a number of different polymeric species in varying proportions, depending on the molar ratio of aluminum to chloride and the conditions employed during manufacture. All such mixtures are employable herein. It is especially desirable to employ what is commonly called activated aluminum chlorohydrate or enhanced activity aluminum chlorohydrate, sometimes abbreviated to AACH, in which the proportion of the more active species, such as Band III species (by a conventional chromatographic method) is higher by virtue of its method of manufacture. In one definition of activated, given in EP 6739, the material has greater than 20% Band III. Other methods of making AACH are given in EP 191628 and EP 451395. AACH is often made by recovery of an aluminum chlorohydrate from a dilute solution under strictly controlled reaction/maturing/dewatering/drying conditions. AACH is commercially available by name, or as activated or enhanced activity, from suppliers such as Reheis, Summit Research and B K Giulini.

Zirconium actives can usually be represented by the empirical general formula: ZrO(OH)_{2n-nz}B_{z}.wH₂O in which z is a variable in the range of from 0.9 to 2.0 so that the value 2n-nz is zero or positive, n is the valency of B, and B is selected from the group consisting of chloride, other halide, sulfamate, sulfate and mixtures thereof. Possible hydration to a variable extent is represented by wH₂O. Preferable is that B represents chloride and the variable z lies in the range from 1.5 to 1.87. In practice, such zirconium salts are usually not employed by themselves, but as a component of a combined aluminum and zirconium-based antiperspirant.

The above aluminum and zirconium salts may have coordinated and/or bound water in various quantities and/or may be present as polymeric species, mixtures or complexes. In particular, zirconium hydroxy salts often represent a range of salts having various amounts of the hydroxy group. Zirconium aluminum chlorohydrate may be particularly preferred.

Antiperspirant complexes based on the above-mentioned astringent aluminum and/or zirconium salts can be employed. The complex often employs a compound with a carboxylate group, and advantageously this is an amino acid. Examples of suitable amino acids include dl-tryptophan, dl-β-phenylalanine, dl-valine, dl-methionine and β-alanine, and preferably glycine which has the formula CH₂(NH₂)COOH.

It is highly desirable in some embodiments of the instant invention to employ complexes of a combination of aluminum halohydrates (especially chlorohydrates) and zirconium chlorohydrates together with amino acids such as glycine, which are disclosed in U.S. Pat. No. 3,792,068 (Luedders et al). Certain of those Al/Zr complexes are commonly called ZAG in the literature. ZAG actives generally contain aluminum, zirconium and chloride with an Al/Zr ratio in a range from 2 to 10, especially 2 to 6, an Al/Cl ratio from 2.1 to 0.9 and a variable amount of glycine. Actives of this preferred type are available from Westwood, from Summit and from Reheis. In one preferred embodiment, the antiperspirant salt is an aluminum zirconium tetrachlorohydrate complex with glycine, herein referred to as AAZG.

It is particularly preferred for the antiperspirant salts to be at least substantially free from aluminum sulfate, by which is meant that the weight proportion of the sulfate present therein is less than 5%, especially less than 3% and particularly less than 1% of the total weight of the antiperspirant salt. Total absence would be very suitable.

Other antiperspirant actives which may be utilized include astringent titanium salts, for example those described in GB 2299506A.

The amount of antiperspirant active present in the compositions of this invention is from 15 to 30 wt. %, with compositions comprising from 18 to 25 wt. % of antiperspirant active being of particular interest. In at least one embodiment of this invention, the antiperspirant stick composition comprises from 20 to 25 wt. % of antiperspirant active.

In at least one embodiment, it is desirable that the mean particle size of the antiperspirant active in the antiperspirant feedstock is in the range of 0.1 to 100 μm, with feedstocks in which 95% by weight of the antiperspirant active particles have a particle size below 50 microns, being of particular interest. In many desirable products, the antiperspirant active, as added, has a mean particle size of from 3 to 30 μm, more particularly from 5 to 25 μm, and in certain highly desirable embodiments from 10 to 25 μm.

Carrier Oil

The carrier oil component of the subject compositions comprises at least one volatile silicone oil and at least one non-volatile, non-silicone, masking oil.

The carrier oils are water immiscible (alternatively describable as hydrophobic or lipophilic) liquids that are liquid at a temperature of 20° C. up to at least the temperature at which the gellant is dissolved or dispersed therein and in which the antiperspirant active, and other particulate materials which may be present, are suspended. It will be recognized that such dissolution temperature depends mutually on the particular gellants or oils. Normally, the oils will have boiling points in excess of 150° C., and often at least 200° C. Melting and boiling point data, as well as information as whether a material is or is not water immiscibile is available from numerous literature sources, for example, the CRC Handbook of Chemistry and Physics published by CRC Press. For any material where such data is not available in the literature, it can be measured simply by any chemist using conventional techniques.

As used herein the term “volatile” is used to designate a material having a measurable vapour pressure at 25° C. Typically the vapour pressure of a volatile oil lies in a range of at least 1 Pa or preferably at least 10 Pa at 25° C., though generally will be less than 4 kPa (30 mmHg). A non-volatile oil can be considered to generate a vapour pressure of below 1 Pa at 25° C.

Volatile silicone oils can be linear or cyclic polyorganosiloxanes or mixtures thereof. Preferred cyclic siloxanes include polydimethylsiloxanes and particularly those containing from 3 to 9 silicon atoms and preferably not more than 7 silicon atoms and most preferably from 4 to 6 silicon atoms, otherwise often referred to as cyclomethicones. Preferred linear siloxanes include polydimethylsiloxanes containing from 3 to 9 silicon atoms. The volatile siloxanes normally by themselves exhibit viscosities of below 10⁻⁵ m²/sec (10 centistokes), and particularly above 10⁻⁷ m²/sec (0.1 centistokes), the linear siloxanes normally exhibiting a viscosity of below 5×10⁻⁶ m²/sec (5 centistokes). The volatile silicone oils can also comprise branched linear or cyclic siloxanes such as the aforementioned linear or cyclic siloxanes substituted by one or more pendant -0-Si(CH₃)₃ groups. Examples of commercially available volatile silicone oils include oils having grade designations 344, 345, 244, 245 and 246 from Dow Corning Corporation; Silicone 7207™ and Silicone 7158™ from Union Carbide Corporation; and SF1202™ from General Electric. In at least one embodiment of particular interest, the volatile silicone oil comprises cyclomethicone.

In the practice of this invention, the volatile silicone oil is present in the stick composition in an amount of from 20 to 40 wt. %, preferably 25 to 35 wt. %. In at least one embodiment of particular interest, the volatile silicone is present in the stick composition in an amount of from 30 to 35 wt. %; in another embodiment of interest the volatile silicone is present in the stick composition in an amount of from 25 to 30 wt. %.

The non-volatile, non-silicone masking oils, alternatively referred to in the description of the invention and claims as non-volatile, non-silicone carrier oils are silicon-free hydrophobic or water-immiscible liquids that are liquid at 20° C. at standard pressure. The non-volatile, non-silicone masking oils are preferably liquid at 15° C., with oils having a boiling point of at least 150° C. being particularly advantageous. Non-limiting examples of the classes of materials from which suitable masking oils can be found include ester oils, ether oils, and hydrocarbon oils. In at least one preferred embodiment, the non-volatile, non-silicone masking oil comprises ester oil, with non-volatile, non-silicone masking oils comprising both ester oil and ether oil being of particular interest.

Ester oils represent a particularly useful class of non-silicone oils. The ester oils can be aliphatic, aromatic or contain both aliphatic and aromatic groups. In one preferred embodiment, ester oil is present in an amount of from 5 to 35 wt. %, more particularly from 5 to 20 wt. %, of the antiperspirant stick composition. In another preferred embodiment ester oil is present in the stick composition in an amount of from 10 to 20 wt. %.

Many desirable aliphatic esters contain at least one long chain hydrocarbon group, for example from 8 to 25 carbons, derived from a monohydric alcohol or mono-carboxylic acid. Suitable aliphatic esters can be derived from monohydric alcohols such as selected from C₁ to C₂₀ alkanols esterified with a carboxylic acid selected from C₈ to C₂₂ mono alkanoic acid and C₆ to C₁₀ alkanedioic acids. Such esters include isopropyl myristate, lauryl myristate, isopropyl palmitate, diisopropyl sebacate and diisopropyl adipate. Other suitable ester oils include glyceride oils and in particular triglyceride oils derived from glycerol and fatty acids, sometimes olefinically unsaturated rather than saturated, containing at least 6 carbons and especially natural oils derived from unsaturated carboxylic acids containing from 16 to 20 and especially 18 carbons. Liquid aromatic ester oils or mixed aromatic/aliphatic ester oils, are preferably derived from benzoic acid. Examples include C₈ to C₁₈ alkyl benzoates or mixtures thereof, including in particular C₁₂ to C₁₅ alkyl benzoates. Many suitable benzoate esters are available under the trademark Finsolv. Other aromatic esters which can be contemplated for use herein comprise double aromatic inclusion. Benzyl benzoate, though feasible, is preferably substantially absent, such as at no more than 5%, and particularly no more than 3% or 1% by weight of the carrier oil blend, and more particularly is excluded. Preferred double aromatic esters comprise a linear or branched alkyl chain, e.g. from 1 to 3 carbons, interposed between ester and/or ether substituted phenyl groups.

As masking agents, aliphatic esters tend to exhibit an intermediate refractive index, and are therefore employed more typically for their emollient properties. Aromatic esters tend to demonstrate a higher refractive index, such as around 1.49 to 1.50 and when double aromatic substitution is present, even a higher refractive index, rendering them particularly suitable for the preparation of translucent compositions containing a particulate astringent antiperspirant salt, and even salts containing zirconium.

Ether oils represent further instances of suitable oils. Preferably, the ether oils contemplatable herein comprise liquid aliphatic ethers derived from a polyglycol especially from polypropylene glycol, PPG, the latter preferably containing at least 3 mers, such as 3 to 20, with a monohydric alcohol. The monohydric alcohol often contains between 3 and 20 carbons. As the molecular weight of the PPG increases, so the chain length of the monohydric alcohol can decrease. Hence, for example, suitable ether oils can vary between a low molecular weight PPG with a long chain fatty alcohol, such as PPG-3 myristyl ether and a lower alkyl ethers of a higher molecular weight PPG, such as the ether named as PPG-14 butyl ether in the CTFA Handbook. In one preferred embodiment, the ether oils comprise from 0 to 30 wt %, of the stick composition, with compositions comprising from 5 to 15% of ether oil being of particular interest.

In many desirable embodiments according to the present invention, the composition comprises at least one ester oil and at least one ether oil, preferably in a weight ratio of ester oil: ether oil of from 5:1 to 1:5, with ester oil:ether oil ratios of from 4:1 to 1:4, more particularly from 3:1 to 1:1, being of particular interest in at least one embodiment. In at least one preferred embodiment, the ratio of ester oil:ether oil is from 2:1 to 1:1, with a ratio of 3:2 being of particular interest.

Suitable hydrocarbon oils are commonly selected from mineral oils, hydrogenated polydecene and hydrogenated polyisobutene. Hydrocarbon oils are desirable in that they, like most of other non-volatile oils described herein, also function as emollients and having a soothing, softening effect on skin.

The non-volatile, non-silicone oils may be natural or synthetic. Natural oils suitable for use herein may take a variety of chemical forms including, for example, glyceryl esters of fatty acids, or triglycerides, normally found in animal and plant tissues, including those which have been hydrogenated to reduce or eliminate unsaturation. However, esters of glycerin and fatty acids may also be synthetically prepared. Many of the natural oils have relatively low refractive indices, i.e., below values of 1.40, and are often employed more for their emolliency than masking ability.

The natural oils employable herein desirably comprise one or more triglycerides of oleic acid, linoleic acid, linolenic acid or ricinoleic acid. Various isomers of such acids often have common names, including linolenelaidic acid, trans 7-octadecenoic acid, parinaric acid, pinolenic acid punicic acid, petroselenic acid and stearidonic acid. It is especially desirable to employ glycerides derived from oleic acid, linoleic acid or petroselenic acid, or a mixture containing one or more of them.

Natural oils containing one or more of such triglycerides include coriander seed oil for derivatives of petroselinic acid, impatiens balsimina seed oil, parinarium laurinarium kernel fat or sabastiana brasilinensis seed oil for derivatives of cis-parinaric acid, dehydrated castor seed oil, for derivatives of conjugated linoleic acids, borage seed oil and evening primrose oil for derivatives of linoleic and linolenic acids, aquilegia vulgaris oil for columbinic acid and sunflower oil, olive oil or safflower oil for derivatives of oleic acid, often together with linoleic acids. Other suitable oils are obtainable from hemp, which can be processed to derive stearadonic acid derivatives and maize corn oil. An especially convenient natural oil by virtue of its characteristics and availability comprises sunflower oil, ranging from those rich in oleic acid glycerides to those rich in linoleic acid glycerides, rich indicating that its content is higher than that of the other named acid.

In one embodiment, the natural oils are present in the compositions of this invention in amounts of from 0 to 3 wt. %, more particularly from 0.1 to 3 wt %., and even more particularly, from 0.5 to 2 wt. %.

The total amount of non-volatile, non-silicone masking oil in the subject compositions is desirably from 20 to 40 wt %, with compositions comprising from 20 to 35 wt % of non-volatile, non-silicone masking oil being of particular interest. In one embodiment of this invention the stick compositions comprise from 20 to 30 weight percent of non-volatile, non-silicone masking oil.

Optionally, the carrier oil may further comprise one or more non-volatile silicone oils. Illustrative, but non-limiting examples of non-volatile silicone oils suitable for use in the practice of this invention are: polyalkyl siloxanes, polyalkylaryl siloxanes and polyethersiloxane copolymers. These can suitably be selected from dimethicone and dimethicone co-polyols. Commercially available non-volatile silicone oils include products available under the trademarks Dow Corning 556 and Dow Corning 200 series, Other non-volatile silicone oils include that bearing the trademark DC704. When present, the non-volatile silicone oil desirably comprises up to 5 wt. % of the stick composition. In at least one embodiment of this invention, the non-volatile silicone oil is present in the stick composition in an amount of from 0.5 to 2 wt. %.

In one preferred embodiment, the total amount of masking oil present in the subject compositions, including both silicone and non-silicone oils, does not exceed 30 wt. %, with compositions comprising a total amount of masking oil of from 20 to 30wt. %, and in some embodiments, from 20 to 25 wt. %, being of particular interest.

Structurant

In the practice of this invention, the structurant functions to gel or solidify the carrier oil. Commonly, the gelation arises by forming a mobile liquid oil phase at an elevated temperature throughout which the structurant is distributed, and in particular by dissolution, such that when the composition cools or is cooled below its setting temperature, a solid product is obtained.

The structurant used herein comprises one or more fatty alcohols that normally are linear and desirably comprise from 16 to 24 carbons, with fatty alcohols comprising from 18 to 22 carbons being of particular interest. Non-limiting examples of suitable fatty alcohols being stearyl alcohol, cetyl alcohol and behenyl alcohol, with stearyl alcohol, being of particular interest in the practice of this invention.

The fatty alcohol is present in the stick compositions of this invention in an amount of at least 12 wt. %, more particularly, at least 15 wt. %. In at least one embodiment of this invention the subject compositions comprise from 15 to 25% wt. %, more particularly from 15 to 20 wt. % of fatty alcohol. In an embodiment of particular interest, C₁₈-C₂₂ fatty alcohols comprise from 90 to 100% by weight, preferably from 95 to 100% by weight of the total fatty alcohol present in the subject compositions.

The structurant further comprises a co-structurant having a melting point of 75 to 95° C., a portion of which comprises polyethylene. Preferably, the co-structurant is a cosmetically acceptable wax having a melting point of from 75-90° C., more particularly, from 80-90° C. In an embodiment of particular interest, the polyethylene has a weight average molecular weight of from 200 to 600, more particularly, from 300 to 500.

The co-structurant is present in the stick composition in amounts of from 2 to 10 wt %, preferably from 3 to 8 wt. %, based on the total weight of the stick, with the polyethylene component thereof being present in an amount of 0.3 to 3 wt. % preferably from 0.5 to 2 wt. %, based on the total weight of the stick. In an embodiment of particular interest the stick composition comprises from about 0.7 to 1.5 wt. % of the polyethylene co-structurant.

In addition to the polyethylene, exemplary, but not exhaustive, of the types of materials from which the co-structurant may be selected are: beeswax, candelilla wax, spermaceti wax, carnauba wax, and other organic materials having similar properties. Such other waxes include natural and synthetic hydrocarbon waxes, e.g., paraffin wax, mineral wax, microcrystalline wax, waxy silicone polymers containing alkyl substituents of at least C₁₀ chain length, waxy components of natural waxes, such as ester components identified in beeswax, be they extracted from natural beeswax, synthesised or modifications to beeswax, including for example, stearyl beeswax or siliconyl beeswax. Other suitable co-structurants include solid ester derivatives of glyceryl or glycol, typically with linear saturated fatty acids, usually containing a significant fraction Of C₁₆₋₂₂ acid residues, which may be synthesized or obtained by hydrogenating the corresponding natural oil, e.g. the glyceride oils described hereinbefore, including castor wax, a particularly preferred co-structurant.

Other co-structurants which can be contemplated include other hydrogenated triglycerides, or aliphatic fatty esters or hydrocarbon waxes having a melting point in the desired melting point range, or blends of two or more of such waxes. These can be readily identified in literature.

In a preferred embodiment the antiperspirant stick compositions of this invention further comprise one or more additional optional components selected from the group consisting of: wash-off agents; skin feel improvers; inorganic particulates; skin benefit agents; colorants; fragrances, preservatives; humectants; and emulsifiers.

In one embodiment of particular interest there is provided an antiperspirant stick composition consisting essentially of:
a) from about 15 to about 30 wt. %, based on the total weight of the stick composition, of antiperspirant active;
b) carrier oil comprising:
   i) volatile silicone oil, the volatile silicone oil being present in an amount of from about 20 to about 40 wt. %; and
   ii) non-volatile, non-silicone masking oil, the non-volatile, non-silicone masking oil being present in the stick composition in an amount of from 20 to 30 wt. %;
c) structurant comprising:
   i) fatty alcohol, the fatty alcohol being present in the stick composition in an amount of from about 15 to about 25 wt. %;
   ii) cosmetically acceptable wax having a melting point of 75 to 90° C., a portion of which comprises polyethylene, the cosmetically acceptable wax being present in the stick composition in an amount of from 3 to 8 wt. %, and the polyethylene being present in the stick composition in an amount of from 0.7-3 wt. %.
      Method of Manufacture

The compositions according to the present invention can be made conveniently in accordance with processes that are typically employed to produce stearyl-alcohol containing solid antiperspirant sticks.

One suitable general method of manufacture of a firm or semi-solid stick comprises the steps of

a) forming a mixture of an oil phase with the structurant dispersed therein;
b) heating the mixture to an elevated temperature at which the gellant becomes molten or dissolved in the oil phase;
c) introducing particulate astringent antiperspirant salt into the oil phase, (step (c) being being carried out before, after or simultaneously with steps (a) or (b));
d) introducing the resultant mixture into a dispenser and
e) cooling or allowing said resultant mixture to cool to below its setting temperature, at least part of this step optionally occurring before step (d).

The temperature to the dispersed mixture is heated in step (b) is typically in the range of from 75 to 95° C. to correspond to melting temperature of the cosmetically acceptable co-structurant wax.

The order of introduction of the other ingredients is at the discretion of the manufacturer. It will be recognized that optional ingredients, if any, can be introduced at a convenient step in the process. Thus, any temperature sensitive ingredient is desirably introduced into the composition shortly before the dispenser is charged, and preferably at a temperature within 10° C. of the setting temperature.

The compositions produced herein are suitable for dispensing from cosmetic dispensers. Such dispensers commonly comprise a barrel, often of round or oval transverse cross section, having an opening at a first end through which the composition is dispensed and an advancing mechanism at an opposed second end that can be used to move the stick composition through the dispenser. Suitable dispensers for firm sticks are described, for example in U.S. Pat. No. 4,232,977, U.S. Pat. No. 4,605,330, WO09818695, WO09603899, WO09405180, WO09325113, WO09305678, EP1040445, U.S. Pat. No. 5,997,202, U.S. Pat. No. 5,897,263, U.S. Pat. No. 5,496,122, U.S. Pat. No. 5,275,496, U.S. Pat. No 6,598,767, U.S. Pat. No. 6,299,369, or WO 2002103830.

The compositions of the present invention can be topically applied to skin, and particularly to underarm skin by extruding the composition in stick form above the top of the barrel and thereafter wiping the stick across the skin surface, thereby depositing a fraction of the composition on the skin. The action can be repeated until the user considers that sufficient composition has been deposited, often in the region of 3 to 8 wipes per armpit. The composition is commonly applied shortly after the armpit has been washed or shaved. The composition is thereafter left in place, conventionally, for a period of time commonly between 5 and 24 hours until it is washed off, usually using soap or a conventional shower gel, and water, for example applied using a flannel, loofah, sponge or even fingers. When seeking to inhibit perspiration, the weight of antiperspirant active applied per armpit is often in the range of from 0. 15 to 0.5 grams.

The following non-limiting examples are provided to further illustrate the invention; the invention is not limited thereto.

### EXAMPLES

As used in the Examples, the designations APS1-APS 30 refer to commercial products that include both stearyl alcohol and non-stearyl alcohol sticks. Not all of the sticks contain antiperspirant active, however, all are fragranced. Based on package information, the sticks are identified as containing ingredients that include the following antiperspirant actives, structurants, oils, and particulates:
APS1: Aluminum zirconium tetrachlorohydrex, PPG-14 butyl ether; castor wax (hydrogenated castor oil), stearyl alcohol, and cyclomethicone.
APS2: Aluminum zirconium tetrachlorohydrex, PPG-14 butyl ether, castor wax (hydrogenated castor oil), stearyl alcohol, and cyclomethicone.
APS3: Aluminum zirconium tetrachlorohydrex glycine, PPG-14 butyl ether, castor wax (hydrogenated castor oil), stearyl alcohol, sunflower seed oil, and cyclomethicone.
APS 4: Aluminum zirconium trichlorohydrex glycine, PPG 14-butyl ether, phenyl trimethicone, stearyl alcohol, castor wax, cyclomethicone, and behenyl alcohol.
APS 5: Non-stearyl alcohol stick (listed ingredients include PPG3-myristyl ether, nylon 6-11/dimethicone copolymer, isostearyl alcohol, dibutyl lauroyl glutamide, and cyclomethicone).
APS 6: Aluminum zirconium tetrachlorohydrex glycine, cyclopentasiloxane, stearyl alcohol, hydrogenated polydecene, and hydrogenated castor oil.
APS 7: Aluminum zirconium tetrachlorohydrex glycine, cyclopentasiloxane, stearyl alcohol, ethylhexyisononanoate, phenyl trimethicone, PPG-14 butyl ether, hydrogenated castor oil, talc, isostearyl behenate₁ polyethylene, PEG-25 propylene glycol stearate, silica, cyclotetrasiloxane, and dimethiconol.
APS 8: Non-stearyl alcohol stick that contained aluminum zirconium tetrachlorohydrex glycine.
APS 9: Non-stearyl alcohol stick that contained aluminium zirconium tetrachlorohydrex glycine.
APS 10: Aluminium zirconium trichlorohydrex glycine, cyclopentasiloxane, stearyl alcohol, hydrogenated castor oil, petrolatum, PPG-14 butyl ether, mineral oil, and behenyl alcohol.
APS 11: Aluminum zirconium trichlorohydrex glycine, cyclopentasiloxane, stearyl alcohol, hydrogenated castor oil, petrolatum, PPG-14 butyl ether, mineral oil, and behenyl alcohol.
APS 12: Aluminum zirconium tetrachlorohydrex glycine, cyclomethicone, stearyl alcohol, PPG-14 butyl ether, hydrogenated castor oil, myristyl myristate, cornstarch, silica dimethyl silylate, and silica.
APS 13: Aluminum zirconium trichlorohydrex glycine, cyclopentasiloxane, stearyl alcohol, phenyl trimethicone, hydrogenated castor oil, petrolatum, silica, and behenyl alcohol.
APS 14: Aluminum zirconium trichlorohydrex glycine, cyclopentasiloxane, stearyl alcohol, phenyl trimethicone, hydrogenated castor oil, petrolatum, silica, and behenyl alcohol.
APS 15: Aluminum zirconium trichlorohydrex glycine, cyclomethicone, stearyl alcohol, mineral oil, hydrogenated castor oil, cetyl alcohol, diisopropyl adipate, aluminium starch octenylsuccinate, PEG-100 stearate and glyceryl stearate, silica, talc, PPG-14 butyl ether.
APS 16: Aluminium zirconium tetrachlorohydrex glycine, cyclomethicone, stearyl alcohol C12-15 alkyl benzoate, hydrogenated castor oil, hydrolyzed corn starch.
APS 17: Aluminium zirconium tetrachlorohydrex glycine, cyclopentasiloxane, stearyl alcohol, PPG-14 butyl ether, phenyl trimethicone, and hydrogenated castor oil.
APS 18: A non-stearyl alcohol stick similar to that of APS 5, and sold under the same brand, but having a different product name.
APS 19: Aluminum zirconium tetrachlorohydrex glycine, cyclomethicone, stearyl alcohol, PPG-14 butyl ether, phenyl trimethicone, and hydrogenated castor oil.
APS 20: Aluminum chlorohydrate, cyclomethicone, stearyl alcohol, PPG-14 butyl ether; talc, hydrogenated castor oil, glyceryl stearate, and octyldodecanol.
APS 21: Aluminum zirconium tetrachlorohydrex glycine, cyclomethicone, stearyl alcohol, PPG-14 butyl ether, talc, hydrogenated castor oil, glyceryl stearate, and octyldodecanol.
APS 22: Cyclomethicone, aluminum zirconium tetrachiorohydrex glycine, stearyl alcohol, PPG-14 butyl ether, phenyl trimethicone, hydrogenated castor oil, cornstarch, polysaccharide.
APS 23: Cyclomethicone, stearyl alcohol, aluminum zirconium tetrachlorohydrex glycine, PPG-14 butyl ether, phenyl trimethicone, and hydrogenated castor oil.
APS 24: Aluminum zirconium trichlorohydrex glycine, cyclopentasiloxane, stearyl alcohol, phenyl trimethicone, hydrogenated castor oil, mineral oil, silica, and behenyl alcohol.
APS 25: Aluminum-free, non-stearyl alcohol deodorant stick.
APS 26: Aluminum zirconium pentachlorohydrex glycine, cyclopentasiloxane, stearyl alcohol, PPG-14 butyl ether, hydrogenated castor oil, C20-40 pareth-10, myristyl myrstate, cornstarch, silica dimethyl silylate, silica, and isopropyl myristyl.
APS 27: Aluminum zirconium tetrachlorohydrex glycine, PPG-14 butyl ether, hydrogenated castor oil, stearyl alcohol, C12-Cl 5 alkyl benzoate, dimethicone, cyclomethicone, steareth-100, and talc.
APS 28: Aluminum zirconium trichlorohydrex glycine, cyclopentasiloxane, stearyl alcohol, dimethicone, petrolatum, PPG-14 butyl ether, hydrogenated castor oil, talc, mineral oil, behenyl alcohol.
APS 29: Aluminum zirconium trichlorohydrex glycine, cyclpentasiloxane, stearyl alcohol, petrolatum, dimethicone, PPG-14 butyl ether, hydrogenated castor oil, talc, mineral oil, and behenyl alcohol.
APS 30: Aluminum zirconium tetrachlorohydrex glycine, cyclopentasiloxane, stearyl alcohol, cyclohexasiloxane, PPG14-butyl ether, phenyl trimethicone, hydrogenated castor oil, corn starch, polysaccharide. A product similar to that of APS 22, and sold under the same brand, but having a different product name.

Solid stick compositions having the formulations described in Table 1 were made using the following standard process. The oils, gellant, and antiperspirant active components were blended together and heated to approximately 90° C., by which time the wax structurants had melted to form an homogenous mixture. The mixture was permitted to cool while maintaining stirring until its temperature had reached about 70° C., whereupon the antiperspirant active was introduced followed by the fragrance. When the mixture reached about 60° C., it was poured into conventional stick dispensers equipped with a platform and twist-up mechanism.

Formulation 6 was compared to antiperspirant stick compositions APS1 through APS 5 using the hardness and whiteness methods set forth below. Hardness and whiteness data is reported in Table 2, with whiteness data being obtained for three separate sets of tests. APS 1 through APS 4 were all stearyl alcohol-containing sticks, APS 5 was a “non-wax” stick that was free of stearyl alcohol. The APS1 through APS5 sticks did not contain polyethylene.

Hardness Method

Stick hardness is measured using a penetrometer (model HPT 7138 from Precision) equipped with a 2.5 gm brass/steel tapered needle (DIN 51-579) on sticks that have been allowed to equilibrate for approximately 8-24 hours at a temperature of 26.7° C. Measurements are taken from 6 different locations on the side of each stick (measurements being taken 0.5 inch (1.3 cm) apart and 0.75 inch (1.9 cm) from the stick edge), with the hardness values being reported as an average of the six measurements. The depth of needle penetration into the stick is recorded to the nearest tenth of a millimeter.

ps Whiteness Method—L*Value

Whitening characteristics are obtained by measuring the change in color of residues deposited onto a dark surface. In this method, the surface of the sticks are cut with a knife to yield a flat surface for application. The sticks are applied by hand, using an even pressure, onto gray 1200 grit sandpaper (Grainger). Three duplicate applications of each product were made. Each application consisted of four 6 inch (15 cm) strokes (2 forward, 2 backward, one stroke covering the other).

The L*value, a measure of lightness (whiteness), was obtained on five sites in each application area. The most uniform areas are used for the measuring sites and either end was avoided because of the stick “footprint” present. Fifteen measurements were obtained for each formulation (five measurements from each of the three applications) with the L*value being reported as a mean value. A higher L*value indicates more whiteness, a lower L*value less whiteness. L*measurements are taken 1 minute and/or 2 hours after application using a Minolta Chromameter set to read in the L*a*b*measuring mode.

In one preferred embodiment, the compositions of this invention have a 2 hour L*value that, at the 90% confidence level, preferably at the 95% confidence level, is statistically less than that of a Test Standard as hereinafter described, in back-to-back comparative Whiteness Method testing. In another preferred embodiment, the compositions of this invention have a 2 hour L*value that is at least 5% less than that of the Test Standard in such testing. Compositions wherein the 2 hour L*value is at least 6% less than that of the Test Standard are of particular interest. As used throughout the specification and claims, the term “2 hour L*whiteness improvement”, refers to a reduction in the 2 hour L*value as compared to the Test Standard in back-toback comparative Whiteness Method testing. For purposes of comparative analysis, the Test Standard is formulated as a composition that contains: 4% castor wax (mp 80° C.), 1% polyethylene (400 mw; mp 82-84° C.), 18% stearyl alcohol, 1% silica, 8% PPG-14 butyl ether, 31% C12-C15 alkyl benzoate (Finsolv TN), 1% 50 cst. dimethicone (Dow Corning 200), 0.5% sunflower oil, 2.5% talc, 0.05% BHT (butylated hydroxytoluene), 8.95% cyclopentasiloxane (Dow Corning 245), and 24% aluminium zirconium tetrachlorohydrex glycine (17.8%). The Test Standard has the same formulation as that of Formulation 11 of Table 1, except that the fragrance oil of Formulation 11 is replaced by an equal amount of cyclopentasiloxane. Notwithstanding this difference, Formulation 11 is expected to perform comparably to the Test Standard in comparative Whiteness Method testing.

TABLE 1
Antiperspirant Formulations

| | FORMULATION NO. | | | | | | | | | | |
|---|---|---|---|---|---|---|---|---|---|---|---|
| COMPONENT (wt. %) | 1 | 2 | 3 | 4 | 5 | 6 | 7 | 8 | 9 | 10 | 11 |
| Castor Wax (mp 80° C.) | 3.5 | 4 | 3.5 | 3.5 | 3.5 | 3.5 | 3.5 | 3.5 | 3.5 | 4 | 4 |
| Stearyl Alcohol (mp 60°) | 18 | 18 | 20 | 18 | 18 | 18 | 18 | 18 | 18 | 18 | 18 |
| Polyethylene | 0.3 | 0.5 | 0.5 | 0.5 | 0.5 | 1 | 1 | 1 | 1 | 1 | 1 |
| (400 mw; mp 82-84° C.) | | | | | | | | | | | |
| Silica | 0.3 | | | 0.3 | 0.3 | | | 0.5 | 0.5 | 0.75 | 1 |
| PPG-14 Butyl Ether | 20 | 5 | 5 | 20 | 20 | 9.5 | 15 | 15 | 25 | 31.25 | 8 |
| C12-C15 Alkyl Benzoate | 5 | 20 | 18 | 5 | 5 | 15 | 23.5 | 23 | 13 | 8 | 31 |
| (Finsolv TN) | | | | | | | | | | | |
| Dimethicone, 50 cst; (Dow | 1 | 1 | 1 | 1 | 1 | 1 | 1 | 1 | 1 | 1 | 1 |
| Corning 200) | | | | | | | | | | | |
| Sunflower Oil | 0.5 | 0.5 | 0.5 | 0.5 | 0.5 | 0.5 | 0.5 | 0.5 | 0.5 | 0.5 | 0.5 |
| Talc | | | | 3.5 | 2 | | | | | | 2.5 |
| AAZG (17.8%) | 24 | 20 | 20 | 20 | 20 | 20 | 24 | 24 | 24 | 24 | 24 |
| Fragrance | 1 | 1 | 1 | 1 | 1 | 1 | 1 | 1 | 1 | 1 | 1 |
| BHT (butylated hydroxytoluene) | 0.05 | 0.05 | 0.05 | 0.05 | 0.05 | 0.05 | 0.05 | 0.05 | 0.05 | 0.05 | 0.05 |
| Cyclomethicone; (Silicone 245; | 26.35 | 29.95 | 30.45 | 26.65 | 28.15 | 30.45 | 12.45 | 12.45 | 12.45 | 10.45 | 7.95 |
| Dow Corning) | | | | | | | | | | | |

TABLE 2
Whiteness (L*Value)/Hardness Data

| | Formulation 6 | APS1 | APS2 | APS3 | APS4 | APS5 |
|---|---|---|---|---|---|---|
| Hardness | 8.8 | 7.8 | 6.7 | 5.0 | 7.3 | 10 |
| Set 1 - Whiteness | | | | | | |
| 1 min. | 40.9 | 41.7 | — | 44.1 | — | — |
| 2 hrs | 37.8 | 42.4 | — | 67.4 | — | — |
| Set 2 - Whiteness | | | | | | |
| 1 min | 36.2 | 33.7 | — | — | 36.6 | 33.7 |
| 2 hrs | 32.9 | 39.5 | — | — | 33.9 | 33.4 |
| Set 3 - Whiteness | | | | | | |
| 1 min | 40.8 | 41.4 | 42.1 | — | — | — |
| 2 hrs | 37.3 | 42.4 | 44.7 | — | — | — |

Of the stick compositions reported in Table 2, Formulation 6, a stick composition as described by the subject by the subject invention, was the softest of the stearyl alcohol-containing sticks. The data demonstrates that as the compositions dried, greater differences in residue coloration were frequently observed. Thus, while Formulation 6 and APSI gave similar results 1 minute after application, as demonstrated by individual set data, over time, Formulation 6 had better anti-whitening performance. Similar results were found comparing Formulation 6 to both APS2 and APS3. APS4, a harder, less depositing stick, had comparable whitening performance to Formulation 6, as did APS5, a stick which lacked stearyl alcohol.

Panelist Testing was carried out on the Formulation 6 stick composition and a variety of commercially available antiperspirant sticks. Some of the sticks were stearyl alcohol-containing sticks, some were not. Test results are reported in Table 3. The testing was conducted as a balanced and randomized Complete Block Design, with 13-15 trained female panelists participating in the test. Each day, two products were evaluated, one on each forearm, with each product tested being rated for payout, number of strokes to a target dose of 0.15 g, whiteness (skin) and whiteness (black fabric), using the following protocols:

Panelist Test Procedures

4 Stoke Weight Loss (Payout)—The test stick is stroked across the inside forearm inside a marked-off, 4 inch×4 inch (10 cm×10 cm) region beneath the elbow crease. The test stick is stroked over the area four times (the first two strokes being a forward stroke and a backward stroke laid down over same; and the third and fourth strokes being a forward stroke and a backward stroke laid down over same; the third and fourth strokes being laid down next to the first and second strokes). The difference in container weight before and after the application of four strokes of product is reported as the 4-Stroke Weight Loss, 4-Stroke Weight Loss is reported as an average of the panelist results

Strokes to Target Dose (0.15 g)—After the initial four stroke application, if a target dose of 0.15 g is not attained, panelists continue to apply product to their forearm (periodically weighing the container following product application, with the container being weighed more frequently as more strokes were applied) until a target of 0.15 g is deposited. Strokes to Target Dose are reported as an average of the panellist results. In one preferred embodiment of this invention, in back-to-back panellist testing, following this Stroke to Target Dose methodology and employing 13 trained female panelists as the test panel, the compositions of this invention have an average Stroke to Target Dose value that does not exceed that of the Test Standard (i.e., the composition described above in the contest of comparative Whiteness Method Testing) by more than 50%. In another preferred embodiment of this invention, the compositions of this invention have an average Stroke to Target Dose value that does not exceed that of the Test Standard by more than 20 percent. In yet another preferred embodiment of interest, the compositions of this invention have an average Stroke to Target Dose value that, at the 95% confidence level is statistically equivalent to or less than that of the Test Standard.

Whiteness (Skin)—After reaching a target dose of about 0.15 g of product, the panelist assigns a ranking between 0 (no whiteness) to 100 (most whiteness) to the applied product. The ranking is determined by standing and holding the test arm at a 30 degree angle to the body. This ranking is given immediately after application (initial) and 5 minutes after application. Whiteness (skin) data is reported as average of the panelist results.

Whiteness (Black fabric)—Immediately after the Whiteness (Forearm Evaluation) a 5 inch×5 inch (13 cm×13 cm) square of black cotton fabric is laid over the forearm test site and, with the test arm folded back towards the shoulder creasing the fabric tightly in the crook of the arm, the square is pulled from this position. Panelists rank the whiteness of the black fabric square on a scale of 0 (no whiteness) to 100 (most whiteness) at intervals of 5 minutes, 10 minutes and 2 hours after the product was collected. Whiteness (black fabric) data is reported as an average of the panelist results.

TABLE 3
Panelist Data

| | | | | | | Whiteness | | |
|---|---|---|---|---|---|---|---|---|
| | | 4-Stroke | | Whiteness | Whiteness | (black | Whiteness | Whitness |
| | No. of | Weight | Strokes to | (skin) | (skin) | fabric) | (black fabric) | (black fabric) |
| FORMULATION | Panelists | Loss (cg) | target dose | Initial | 5 Min | 5 Min | 10 min | 2 Hr |
| APS 6 | 15 | 11.7E | 7.5CD | 8.1DEFG | 9.5DEF | 27.3DE | 35.7DEF | 54.1D |
| APS 7 | 15 | 12.9DE | 5.6DEFG | 14.1ABC | 13.3CDE | 24.3DEF | 28.5FG | 36.6E |
| APS 8 | 14 | 7.3G | 15.2B | 2.8GH | 31.4A | 55.6A | 61.3AB | 67.3ABC |
| APS 9 | 14 | 5.9H | 24.7A | 1.6H | 21.8B | 60.4A | 67.2A | 70.3A |
| APS 10 | 13 | 15.2C | 4.5FG | 12.5BCD | 14.8BCD | 32.2CD | 40.2DE | 67.8AB |
| APS 11 | 15 | 15.3C | 4.4FG | 9.1CDEF | 11.6CDEF | 28.8CDE | 41.0DE | 66.5ABC |
| APS 12 | 14 | 9.9F | 8.6C | 9.2CDEF | 9.9DEF | 32.4CD | 41.8DE | 60.7ABCD |
| APS 13 | 14 | 15.8C | 4.3FG | 10.4CDE | 8.8DEF | 22.9EF | 29.9FG | 56.2BCD |
| APS 14 | 13 | 18.6B | 4.0G | 10.5CDE | 10.5CDE | 24.6DEF | 31.9EF | 55.9CD |
| APS 15 | 14 | 11.6E | 6.5CDEFG | 6.2EFGH | 8.0DEF | 24.6DEF | 30.1FG | 35.2E |
| APS 16 | 15 | 10.0F | 8.1C | 9.9CDE | 11.4CDEF | 28.1CDE | 36.8DEF | 61.4ABCD |
| APS 17 | 14 | 7.5G | 16.B | 5.EFGH | 4.4F | 18.2FG | 21.6GH | 36.2E |
| APS 18 | 15 | 21.33A | 4G | 2.6GH | 3.4F | 17.6FGH | 20.8GH | 24.3F |
| Formulation 6 | 14 | 11.5E | 7.1CDE | 5.9EFGH | 5.5EF | 14.3GH | 20.1GH | 23.0F |

Entries sharing a letter are not significantly different at the 95% confidence level.

Of the sticks reported in Table 3, APS 18 (a substantially harder, non-stearyl alcohol stick) and Formulation 6 were the least whitening sticks over time.

Formulation 6 was tested against a variety of commercially available stick products in another series of panellist tests, following the Panellist Testing Procedures described above. Test results are reported in Table 4.

TABLE 4
Panelist Data

| | | | | | | Whiteness | | |
|---|---|---|---|---|---|---|---|---|
| | | 4 Stroke | | Whiteness | Whiteness | (Black | Whiteness | Whitness |
| | No. of | Weight | Strokes to | (Skin) | (Skin) | fabric) | (Black fabric) | (Black fabric) |
| FORMULATION | Panelists | Loss (cg) | target dose | Initial | 5 Min | 5 Min | 10 min | 2 Hr |
| Formulation 6 | 15 | 10.5B | 9.9DE | 6.9BC | 6.8CD | 20.1DE | 23.5DEF | 28.0G |
| APS 19 | 13 | 6.5DE | 23.2A | 6.2C | 5.5D | 18.3DE | 18.6F | 36.2FG |
| APS 20 | 14 | 10.8B | 8.3DEF | 11.6AB | 13.1AB | 42.0A | 51.3A | 71.9A |
| APS 21 | 15 | 6.1E | 25.5A | 7.3BC | 7.6CD | 25.9CDE | 38.4B | 59.4B |
| APS 22 | 13 | 8.5C | 11.6CDE | 6.1C | 5.8D | 19.9DE | 25.3CDEF | 47.7CDE |
| APS 23 | 15 | 7.9CD | 17.5B | 6.1C | 5.6D | 17.3E | 22.3EF | 37.6EFG |
| APS 24 | 14 | 13.3A | 5.5F | 8.4ABC | 11.4ABC | 28.8BC | 33.6BC | 56.1BC |
| APS 25 | 13 | 8.0C | 14.8BC | 8.9ABC | 8.2BCD | 26.7CD | 32.2BCD | 51.3BC |
| APS 6 | 13 | 8.5C | 12.2CD | 8.3ABC | 8.8BCD | 23.7CDE | 31.3BCDE | 55.3BC |
| APS 26 | 15 | 14.1A | 5.1F | 13.4A | 15.2A | 36.3AB | 47.1A | 70.5A |
| APS 3 | 14 | 14.5A | 4.6F | 8.1BC | 10.9ABC | 26.6CD | 35.4B | 50.5BCD |

Entries sharing a letter are not significantly different at the 95% confidence level.

Of the sticks reported in Table 4, Formulation 6 had the best overall combination of desirable payout and low whiteness (skin and black fabric). APS 19 and APS 23 exhibited low overall whiteness, but were significantly harder sticks with lower payouts.

Solid stick compositions having the formulations described in Table 5 were prepared following the procedure described above for Formulations 1 to 11.

TABLE 5
Antiperspirant Stick Formulations

| | FORMULATION NO. | | | | | | | |
|---|---|---|---|---|---|---|---|---|
| COMPONENT (wt. %) | 12 | 13 | 14 | 15 | 16 | 17 | 18 | 19 |
| Castor Wax (mp 80° C.) | 3.5 | 3.5 | 3.5 | 3.5 | 3.5 | 3.5 | 3.5 | 3.5 |
| Stearyl Alcohol (mp 60°) | 18 | 18 | 18 | 18 | 18 | 18 | 18 | 18 |
| Polyethylene | 1 | 1 | 1 | 1 | 1 | 1 | 1 | 1 |
| (400 mw; mp 82-84° C.) | | | | | | | | |
| Steareth 100 | 0.5 | 0.5 | 0.5 | 0.5 | — | — | 0.5 | — |
| PPG-14 Butyl Ether | 9.5 | 9.5 | 9.5 | 9.5 | 9.5 | 9.5 | 9.5 | 9.5 |
| C12-C15 Alkyl Benzoate | 15 | 15 | 15 | 15 | 15 | 15 | 15 | 15 |
| (Finsolv TN) | | | | | | | | |
| Dimethicone, 50 cst | 1 | 1 | 1 | 1 | 1 | 1 | 1 | 1 |
| (Dow Corning 200) | | | | | | | | |
| Sunflower Oil | 0.5 | 0.5 | 0.5 | 0.5 | 0.5 | 0.5 | 0.5 | 0.5 |
| AAZG (17.8%) | 20 | 20 | 20 | 20 | 30 | 24 | 20 | 24 |
| Fragrance | 1.25 | 1.25 | 1.25 | 1.25 | 1 | 1 | 1.25 | 1 |
| Fragrance Encapsulate | | | | | | | 1.5 | 1.5 |
| BHT (butylated hydroxytoluene) | 0.05 | 0.05 | 0.05 | 0.05 | 0.05 | 0.05 | 0.05 | 0.05 |
| Cyclomethicone (Silicone 245; | 28.7 | 27.7 | 28.2 | 29.7 | 20.45 | 26.45 | 28.2 | 24.95 |
| Dow Corning) | | | | | | | | |
| Cyclodextrin | 1 | 2 | 1.5 | | | | | |

Stick compositions as described in Tables 1 and 5 were evaluated for L*Value, payout, yield stress, and hardness using the following procedures.

| | |
|---|---|
| Whiteness Method | As described above. |
| 4-Stroke | As described above, except that only a single |
| Weight Loss | user carried out the testing. Data is reported as an |
| | average of two trails. |
| Stroke to | As described above, except that only a single user |
| Target Dose | carried out the testing. Data is reported as an |
| (0.15 g) | average of two trials. |
| Yield Stress | Yield stress is preformed on a Bohlin Controlled |
| | Stress rheometer at 50° C. The measurement is |
| | made using the C14 bob and cup geometry. The |
| | temprature of the cup is first heated to 50° C. |
| | The sample to be measured is collected from the |
| | antiperspirants stick test specimen as a cylinder |
| | plug that fit inside the measuring cup. The plug is |
| | collected using a 5 cc syringe (Norm-Ject Syringes, |
| | VWR Cat. No. 53548-010). The tip or the luer and |
| | the end of the syringe are cut with a tube cutter, |
| | which enable the opening to be the same diameter |
| | as the barrel. A 3 cc sample plug is inserted into |
| | the cup and equilibrated at 50° C. for half an hour. |
| | The cup is sealed to prevent solvent evaporation |
| | during this heating period. In the meantime, the |
| | bob is placed in a 50° C. oven to maintain the |
| | same temprature as the sample. After half an hour, |
| | the bob at 50° C. is inserted in the softened |
| | sample and the yield stress measurement is carried |
| | out. To guard against a drying effect, a solvent trap |
| | supplied by Bohlin is used during the measurement. |
| | The stress is applied incrementally from 0.15 Pa to |
| | 500 Pa at 30-second interval. |
| (hardness) Method | As described above |

The results are reported in Table 6.

TABLE 6
L* Value and Other Data

| (L*Value) | 1 | 2 | 3 | 4 | 5 | 6 | 7 | 8 | 9 | 10 | 11 | 12 | 13 | 14 | 15 | 16 | 17 | 18 | 19 |
|---|---|---|---|---|---|---|---|---|---|---|---|---|---|---|---|---|---|---|---|
| 1 min | 38.1 | 38.0 | 38.1 | 38.2 | 38.4 | 38.4 | 38.0 | 37.9 | 37.9 | 38.2 | 37.7 | 38.6 | 38.9 | 38.9 | 38.4 | 38.6 | 38.6 | 39.3 | 40.3 |
| 2 hrs | 34.7 | 34.5 | 34.5 | 34.8 | 34.7 | 34.7 | 36.7 | 37.0 | 37.1 | 37.1 | 37.5 | 35.2 | 35.5 | 35.3 | 35.1 | 35.1 | 34.5 | 35.4 | 35.7 |
| 4 Stroke | 0.11 | 0.07 | 0.05 | 0.04 | 0.05 | 0.07 | 0.06 | 0.08 | 0.10 | 0.08 | 0.08 | 0.12 | 0.07 | 0.08 | 0.08 | 0.10 | 0.07 | 0.08 | — |
| Weight | | | | | | | | | | | | | | | | | | | |
| Loss (g) | | | | | | | | | | | | | | | | | | | |
| Strokes | 8 | 12 | 12 | 14 | 19 | 10 | 11 | 9 | 9 | 8 | 10 | 5 | 7 | 6 | 9 | 5 | 7 | 7 | — |
| To | | | | | | | | | | | | | | | | | | | |
| Target | | | | | | | | | | | | | | | | | | | |
| Dose | | | | | | | | | | | | | | | | | | | |
| Yield | 24 | 21 | 11 | 89 | 36 | 19 | 14 | 18 | 21 | 36 | 27 | — | — | — | 12 | 23 | 14 | — | — |
| Stress | | | | | | | | | | | | | | | | | | | |
| Hardness | 6.2 | 7.2 | 6.0 | 5.8 | 5.3 | 7.8 | 6.0 | 6.8 | 6.6 | 5.1 | 5.2 | 5.5 | 5.5 | 5.7 | 7.8 | 5.2 | 6.5 | 6.6 | — |
| Run 1 | | | | | | | | | | | | | | | | | | | |
| Hardness | 5.7 | 7.4 | 6.4 | 6.2 | 6.1 | 7.5 | 7.2 | 7.1 | 6.5 | 6.2 | 5.3 | 7.1 | 7.2 | 7.7 | 8.0 | 5.3 | 6.8 | 7.3 | — |
| Run 2 | | | | | | | | | | | | | | | | | | | |

In terms of their 2 hour whitening data, stick Formulations 1 to 6 and 12 to 19 were generally less whitening than comparative Formulations 7 to 11. While comparative Formulations 7 to 11 had higher AAZG levels (24 wt %) than Formulations 2-6, 12-15 and 18 the antiperspirant salt levels of Formulations 1, 16, 17 and 19 were the same or higher than those of comparative Formulations 7 to 11. While stick Formulations 4 and 5 had somewhat lower salt levels (20%, compared to 24% for Formulations 7 to 11), they also contained varying amounts of talc (absent from Formulations 7 to 10, but present in Formulation 11).

In another series of tests, additional Whiteness Method Hardness and 4-Stroke Weight Loss testing was conducted following the procedures described above. Hardness data is reported as an average of two trials. 4-Stroke Weight Loss testing was carried out by a single user. Data from this testing is reported in Tables 7 and 8.

TABLE 7

| | | | | 4 Stroke | 4 Stroke |
|---|---|---|---|---|---|
| | | | | Weight | Weight |
| | | | | Loss | Loss |
| | L* | L* | | (cg) | (cg) |
| COMPOSITION | 1 min | 2 hr | Hardness | Run 1 | Run 2 |
| Formulation 15 | 39.0 ± 0.2 | 35.5 ± 0.3 | 7.7 | 14 | 11 |
| APS 28 | 40.3 ± 0.3 | 44.6 ± 2.5 | 6.8 | 13 | 15 |
| APS 29 | 41.1 ± 0.7 | 42.8 ± 1.4 | 7.5 | 19 | 12 |
| APS 6 | 39.4 ± 0.2 | 49.0 ± 1.5 | 8.2 | 8 | 11 |
| APS 30 | 39.0 ± 0.3 | 38.4 ± 0.7 | 8.1 | 14 | 10 |
| APS 11 | 41.2 ± 0.4 | 57.7 ± 1.2 | 6.9 | 15 | 19 |

Formulation 15 had the best two hour whitening performance (least whitening) of the Table 7 sticks.

TABLE 8

| | L* | L* |
|---|---|---|
| COMPOSITION | 1 min | 2 hr |
| Formulation 6 | 39.0 ± 0.3 | 35.4 ± 0.2 |
| Formulation 11 | 38.2 ± 0.2 | 37.7 ± 0.3 |
| APS 27 | 39.0 ± 0.3 | 39.8 ± 0.5 |
| APS 28 | 39.6 ± 0.3 | 42.8 ± 1.3 |
| APS 29 | 40.6 ± 0.3 | 41.3 ± 1.3 |
| APS 6 | 39.4 ± 0.3 | 48.5 ± 0.8 |
| APS 30 | 39.2 ± 0.3 | 37.7 ± 0.7 |
| APS 11 | 40.9 ± 0.4 | 57.3 ± 1.1 |

Formulation 6 had the best two hour whitening performance of the Table 8 sticks.

What is claimed is:

1. An antiperspirant stick composition comprising:
a) from 15 to 30 wt. % antiperspirant active;
b) carrier oil comprising:
i) volatile silicone oil, the volatile silicone oil being present in the stick composition in an amount of from 20 to 40 wt. %;
ii) non-volatile, non-silicone masking oil, the non-volatile, non-silicone masking oil being present in the stick composition in an amount of from 20 to 40 wt. %,
c) structurant comprising:
i) fatty alcohol, the fatty alcohol being present in the stick composition in an amount of from 12 to 25 wt. %;
ii) cosmetically acceptable wax having a melting point of from 75 to 95° C., a portion of which comprises polyethylene, the cosmetically acceptable wax being present in the stick composition in an amount of from 2 to 10 wt. %, and the polyethylene being present in the stick composition in an amount of from 0.3 to 3%.

2. An antiperspirant stick composition as described in claim 1 wherein the fatty alcohol comprises C18-C22 fatty alcohol.

3. An antiperspirant stick composition as described in claim 2 wherein the fatty alcohol comprises stearyl alcohol.

4. An antiperspirant stick composition as described in claim 1 wherein C18-C22 fatty alcohol comprises from about 90 to 100% by weight of the fatty alcohol present in the stick composition.

5. An antiperspirant stick composition as described in claim 1 wherein the volatile silicone is cyclomethicone.

6. An antiperspirant stick composition as described in claim 1 wherein the polyethylene component of the cosmetically acceptable wax has a weight average molecular weight of from 300 to 500.

7. An antiperspirant stick composition as described in claim 1 wherein the cosmetically acceptable wax comprises castor wax and polyethylene.

8. An antiperspirant stick composition as described in claim 1 wherein the fatty alcohol is present of from to 15 to 20 wt. %, based on the total weight of the stick.

9. An antiperspirant stick composition as described in claim 1 wherein the non-volatile non-silicone masking oil comprises an ester oil.

10. An antiperspirant stick composition as described in claim 9 wherein the non-volatile non-silicone masking oil further comprises an ether oil.

11. An antiperspirant stick composition as described in claim 9 wherein the ester oil comprises C8-C18 alkyl benzoate.

12. An antiperspirant stick composition as described in claim 1 wherein the non-volatile, non-silicone masking oil is present in the stick composition in an amount of from 20 to 30 wt. %.

13. An antiperspirant stick composition as described in claim 10 wherein the ether oil comprises PPG 14 butyl ether.

14. An antiperspirant stick composition as described in claim 1 which has a Hardness Method hardness value of from 8 to 10.

15. An antiperspirant stick composition as described in claim 1 that further comprises one or more additional optional components selected from the group consisting of: wash-off agents; skin feel improvers; inorganic particulates, skin benefit agents; colorants; fragrances, preservatives; humectants; and emulsifiers.

16. An antiperspirant stick composition as described in claim 1 wherein the fatty alcohol and polyethylene combined comprise from 95% to 100% by weight of the structurant.

17. An antiperspirant stick composition consisting essentially of:
a) from about 15 to about 30 wt. % of antiperspirant active;
b) carrier oil comprising:
i) volatile silicone oil, the volatile silicone oil being present in the stick composition in an amount of from about 20 to about 40 wt. %; and
ii) non-volatile, non-silicone masking oil, the non-volatile, non-silicone masking oil being present in the stick composition in an amount of from 20 to 30 wt. %;
c) structurant comprising:
i) fatty alcohol, the fatty alcohol being present in the stick composition in an amount of from about 15 to about 25 wt. %;
ii) cosmetically acceptable wax having a melting point of 75 to 90° C., a portion of which comprises polyethylene, the cosmetically acceptable wax being present in the stick composition in an amount of from 3 to 8 wt. %, and the polyethylene being present in the stick composition in an amount of from 0.7-3 wt. %.

18. A method of producing a stick composition according to claim 1 which comprises the steps of:
a) forming at an elevated temperature, a fluid mixture comprising the antiperspirant active suspended in the carrier oil in which the structurant is dispersed and/or dissolved;
b) cooling or permitting the fluid mixture to cool to a temperature at which the mixture sets to form the stick composition.

19. A method of ameliorating perspiration by the topical application to the skin of a composition as described by claim 1.

20. An antiperspirant composition according to claim 1 that has a 2 hour L*whiteness improvement of at least 5%.

21. An antiperspirant composition according to claim 1 having a 2 hour L*whiteness improvement that is statistically significant at the 95% confidence level.

22. An antiperspirant composition according to claim 1 that has an average Stroke to Target Dose value that does not exceed that of the Test Standard by more than 20 percent.
